(19) United States
(12) Patent Application Publication
(10) Pub. No.: US 2008/0193961 A1
(43) Pub. Date: Aug. 14, 2008

(54) Localized Control of Thermal Properties on Microdevices and Applications Thereof

(76) Inventors: Christopher J. Easley, Madison, TN (US); James P. Landers, Charlottesville, VA (US); Jerome P. Ferrance, Charlottesville, VA (US)

Correspondence Address: BLANK ROME LLP, 600 NEW HAMPSHIRE AVENUE, N.W., WASHINGTON, DC, 20037, US

(21) Appl. No.: 11/664,297

(22) PCT Filed: Sep. 29, 2005
(86) PCT No.: PCT/US05/34674

§ 371 Date: Nov. 9, 2007

Related U.S. Application Data
(60) Provisional application No. 60/614,304, filed on Sep. 29, 2004.

(51) Int. Cl. C12Q 1/02; B44C 1/22; B29C 35/08; G01N 1/00; G01N 27/26; B01J 19/00; B05D 3/10; G01N 1/28

(52) U.S. Cl. 435/29; 216/83; 264/400; 73/863.11; 427/307; 436/174; 422/68.1; 204/600

(57) ABSTRACT

The present invention relates to microfluidic devices (20), and in particular, heat management in such devices. To achieve desired thermal properties in selected areas of a microfluidic or nanofluidic device, selective removal or addition of material (thermal mass) can be effected in certain selected regions of the device to control thermal properties, wherein the selected regions are immediately surrounding a reaction chamber (14) and resulting in an empty space (18). This is particularly useful in accommodating rapid heating and/or cooling rates during sample processing and analysis on a microfluidic or nanofluidic device.

This application claims the priority of U.S. Provisional Patent Application Ser. No. 60/614,304, filed Sep. 29, 2004, the disclosure of which is incorporated herein by reference.

### FIELD OF THE INVENTION

The present invention relates to microfluidic devices, and in particular, heat management in such devices.

### BACKGROUND OF THE INVENTION

Miniaturization of analytical methodology onto microdevices has seen a surge of research interest over the recent decade due to the possibilities of reduced reagent and sample volumes, reduced analysis times, and parallel processing. Another leading advantage of miniaturization is the potential to integrate multiple sample handling steps with analysis steps to achieve integrated, user-friendly, sample-in/answer-out devices—commonly referred to as micro-total-analysis systems (μ-TAS).

Microfluidic devices are known. For example, U.S. Pat. No. 6,130,098 to Handique; U.S. Pat. No. 6,919,046 to O'Connor et al.; U.S. Pat. No. 6,544,734 to Briscoe et al.; the disclosures of which are incorporated herein by reference, discloses microfluidic devices for use in biological and/or chemical analysis. The system includes a variety of microscale components for processing fluids, including reaction chambers, electrophoresis modules, microchannels, detectors, valves, and mixers. Typically, these elements are microfabricated from silicon, glass, ceramic, polymer, metal, and/or quartz substrates. The various fluid-processing components are linked by microchannels, through which the fluid flows under the control of a fluid propulsion mechanism. If the substrate is formed from silicon, electronic components may be fabricated on the same substrate, allowing sensors and controlling circuitry to be incorporated in the same device. These components can be made using conventional photolithographic techniques, as well as with laser ablation, polymer molding, hot embossing, micromachining, physical/mechanical removal, or similar methods. Multi-component devices can be readily assembled into complex, integrated systems. In most microfluidic research laboratories, photolithography and chemical etching are used in their simplest form to create patterns in a monolithic configuration. However, as the object of the present invention, it was recognized that with a few alterations these same procedures were ideal for removal of thermal mass to alter the heat dissipation rates.

Photolithography technology developed for the semiconductor industry was, for the most part, easily transferable to fluidic microchip fabrication using electrically insulating substrates such as glass or fused silica. Generally, a printed photomask is used to transfer channel designs onto positive photoresist using UV light, the photoresist is developed, and microchannels are etched into the substrate at the exposed regions using a dilute solution of hydrofluoric acid (HF). A cover plate is then bonded at high temperature (500 to 1100° C.) to the etched plate to create closed fluidic channels, typically on the order of tens to hundreds of micrometers. A sample photomask pattern and resultant microchip is shown by FIGS. 1 and 4, where the channels were etched to a depth of 125 μm.

In performing analysis on microfluidic devices, the thermal properties of each segment of the device then become a critical issue, particularly when different processing or analysis steps require large differences in thermal events. The ability to control these thermal properties could be extremely advantageous.

For instance, for electrophoretic separations, higher applied voltages typically translate into faster separations and better resolution, but the occurrence of Joule heating poses an upper limit on the applied voltage. Electrophoresis microchips with small channels can dissipate heat more quickly than capillaries due to the increase in thermal mass surrounding the microchannel, thereby increasing the heat transfer rate. It should be possible to apply higher field strengths to microscale separations as compared to conventional CE. Through design of the injection channels and use of high field strengths (53 kV cm⁻¹), Jacobson et al. (Anal Chem. 1998, 70, 3476-3480) were able to separate a binary mixture in only 0.8 ms. These microchip thermal properties are favorable for most separations, where higher field strengths can be applied to reduce analysis times.

Although the increased thermal dissipation rates are favorable for microchip electrophoretic separations, they are conversely detrimental to other processes that are desired on these microchips. As mentioned previously, to take fall advantage of the microchip platform, sample processing steps can be integrated with the analytical separation onto a single microdevice. Many sample processing steps—including labeling reactions, synthetic preparation, biochemical reactions, and cell lysis—are temperature-dependent. In situations where rapid temperature increases are not only advantageous but required, the relatively large thermal conductivity and large thermal mass (relative to solution) of these microchips will be unfavorable. This would include any on-chip reaction that required maintenance of an elevated temperature. One example is the temperature cycling required for DNA amplification via the polymerase chain reaction (PCR), which normally takes a few hours to complete by conventional heating block methods and requires that the solution be held at elevated temperatures (94° C.) for a portion of that time. Giordano et al. (Anal Biochem 2001, 291, 124-132) have shown that PCR carried out in polymeric microchips (low thermal conductivity) could be completed in as little as 4 minutes using a non-contact infrared tungsten lamp for heating and forced air cooling. However, these polymer chips are not amenable to other processing or analysis steps. Moving the PCR to glass microchips showed a marked decrease in heating rates due to the increased thermal conductivity of these devices, and amplification times were typically on the order of 30-45 minutes. Therefore, there remains a need for a microfluidic or nanofluidic device that can accommodate rapid heating and/or cooling rates to increase the throughput of the system.

### SUMMARY OF THE INVENTION

Applicant has recognized that selective removal or addition of material (thermal mass) in certain regions of interest on the microfluidic or nanofluidic device is a valuable tool for controlling thermal properties of a microfluidic or nanofluidic device. This is particularly useful in accommodating rapid heating and/or cooling rates during sample processing and analysis on a microfluidic or nanofluidic device. “Thermal mass” as used herein refers to any material that affect thermal conductance in a microfluidic or nanofluidic device. Removal or addition of “thermal mass” can increase or decrease thermal conductance depending on the material used; therefore, “thermal mass” does not necessarily imply a specific effect on heating or cooling rates, only that these rates are affected. Moreover, throughout the application “microfluidic or nanofluidic device,” “microchip,” and “chip” are used synonymously.

It was recognized that removal of thermal mass can be accomplished through minimal modification of current processes. For example, on a glass substrate, using HF-resistant tape as a secondary mask, glass could be selectively removed in any region where a decrease in heat dissipation was needed. The primary photomask could be designed to control the etch depth and, hence, the thickness of the remaining glass layers. To expedite the process, 48% HF solution was used for etching in the glass removal step, which was easily integrated into the normal chip fabrication process. It was therefore possible to utilize existing technology and reagents to achieve further control over the thermal properties of these microchips.

A simple mathematical treatment of the process was developed to confirm the usefulness of the glass removal for decreasing heat dissipation. Although isotropically-etched channels have a trapezoidal cross-section, the microchannel, surrounding glass, and surrounding air can be approximated by a multilayer cylinder (FIG. 2a) with specified boundary conditions as treated by Chapman (In Heat Transfer, 4 ed.; Macmillan Publishing Company: New York, 1974, pp 35-86). Using this treatment, the heat flow per unit length from the channel is defined as

$$\frac{q}{L} = \frac{2 π ( {T}_{1} - {T}_{3} )}{\frac{ln ( {r}_{glass} / {r}_{sol} )}{{k}_{glass}} + \frac{ln ( {r}_{air} / {r}_{glass} )}{{k}_{air}}}$$

where q is the dissipated heat in Joules; L is the length in meters; T₁ and T₃ are the fixed temperatures (in ° C.) of the solution and outer boundary, respectively; rₛₒₗ, r_{glass}, and rₐᵢᵣ are the surface radii (in meters) of the solution, glass, and air layers, respectively; and k_{glass} and kₐᵢᵣ are the thermal conductivities of the glass and air layers, respectively. Using the ratio of the heat flow at variable r_{glass}, to the heat flow at a fixed r_{g0}≧r_{glass} and rₐ₀≧rₐᵢᵣ, the temperature difference term is eliminated, leaving a heat flow ratio (“FR) equation

$$HFR = \frac{\frac{ln ( {r}_{g 0} / {r}_{sol} )}{{k}_{glass}} + \frac{ln ( {r}_{a 0} / {r}_{g 0} )}{{k}_{a 0}}}{\frac{ln ( {r}_{glass} / {r}_{sol} )}{{k}_{glass}} + \frac{ln ( {r}_{air} / {r}_{glass} )}{{k}_{air}}}$$

If the constants are defined by typical microchip values and the HFR is plotted versus r_{glass} in the range 0>r_{glass}>r_{g0}, the heat flow is shown to increase sharply at first, then more linearly approach a value of 1.0 (FIG. 2b). This theoretical result indicates that removal of the heat conducting glass (thermal mass) from around the microchannel decreases the rate of heat loss to the surroundings, as expected. In fact, these equations show a possible 4.2-fold decrease in heat loss by etching the surrounding glass thickness from 1.1 mm to 0.05 mm. Note that this treatment assumes a capillary geometry for simplicity, while microchannels normally have trapezoidal cross-section and are etched into glass plates. These glass plates are expected to show an even farther decrease in heat loss under the same conditions. However, the present inventors have unexpectedly discovered that the removal of thermal mass actually assists in increasing the cooling rate in convective surroundings, especially when used with forced air cooling.

An object of the present invention is to control heat transfer in selected areas of a microfluidic or nanofluidic device.

Another object of the present invention is to provide a method for increasing throughput for analyses carried out on microfluidic or nanofluidic devices.

Another object of the present invention is to provide a microfludic devices having structures that are capable of increased heating and/or cooling rates, and methods of making thereof.

Another object of the present invention is to provide methods for performing rapid analyses on microfluidic or nanofluidic devices.

To accomplish the objects of the present invention, thermal mass is removed from or added to selected areas on a microfluidic or nanofluidic device. This is accomplished by identifying areas on the microfluidic or nanofluidic device where rapid heating and/or cooling, or extra insulation is desired; and selectively removing or adding materials (thermal mass) surrounding the selected areas without destroying the integrity of the microscale components (e.g. reaction chambers, electrophoresis modules, microchannels, detectors, valves, or mixers) of the microfluidic or nanofluidic device. The removal of materials may be completely through the microfluidic or nanofluidic device or partially. The removal process must also maintain the functional and structural integrity of the microscale component and the microfluidic or nanofluidic device.

The heat dissipation rates of a microfluidic or nanofluidic device can be altered in regions of interest by chemical removal of thermal mass, with the location of these regions being easily controlled, e.g., by mask patterns. Other regions of the microfluidic or nanofluidic device, in which it is not preferable to alter heat dissipation (e.g. separation domain), can be geometrically isolated to reduce any or all effects of the removal. Furthermore, the reagents and expertise necessary for this process are already part of the standard microchip fabrication steps. The current method can be accomplished by only adding a few simple steps to existing methods for making microfludic devices.

Even further, any situation in which spatial thermal control is necessary can benefit from the present invention. It is clear that more rapid thermal cycling can be achieved where desired, but even in settings that need only maintain the temperature at a single value, the total power consumption can be decreased by insulating the heated region using the procedures of the present invention. Insulation can be added to desired areas of the microfluidic or nanofluidic device by increasing the thickness of material (thermal mass) is those areas. This approach is essentially the opposite for those regions where thermal mass is removed for improved heating and/or cooling rates (increasing thermal mass rather than removing thermal mass). The thickness can be increased by adding the same or different material to selected regions on the microfluidic or nanofluidic device. The material is selected to achieve the desired thermal effect. For example, an insulating polymer can be added to increase insulation in the selected areas; or a highly conductive metal can be added to increase thermal conductivity. The general approach should be applicable to any or all other substrates (glass, ceramic, various polymers (such as plastics), metal, silicon, quartz, etc.) using any number of mass removal procedures (etching, laser ablation, polymer molding, hot embossing, micromachining, physical/mechanical removal, etc.).

The present invention allows for the ability to achieve localized control of thermal properties on fluidic microchips. Independent of substrate or removal procedure, the deliberate removal of thermal mass in specific regions can alter the thermal properties of those regions, providing a means of thermal control through fabrication. In most cases, the removal procedure can simply be a modified version of the standard procedure used to create structures on the microfluidic or nanofluidic device, thereby minimizing the added fabrication costs. The particular substrate outlined here is borosilicate glass, with the mass removal procedure being chemical etching with hydrofluoric acid (HF); however, other substrates (e.g., ceramics, various polymers, silicon, metals, or quartz) and removal process (e.g., etching, laser ablation, polymer molding, hot embossing, micromachining, or physical/mechanical removal) are also appropriate. Because glass is the prevailing substrate in microfluidics research, localized control of thermal properties on these devices is of considerable importance. For example, integration of chemical or biochemical reactions onto these devices plays a fundamental part in the development of micro-total analysis system (μ-TAS). The thermal properties of reaction chambers could feasibly be tailored to specific reactions using the present invention, thereby maximizing the reaction yield while reducing the time of reaction necessary. This type of localized thermal control can be applied to any number of functionalities on microchips to enhance performance.

### BRIEF DESCRIPTION OF THE DRAWINGS

FIG. 1a shows a photograph of a glass microfluidic device.

FIG. 1b shows a schematic drawing of the glass microfluidic device photographed in FIG. 1b.

FIG. 2a is a schematic of a multilayer cylinder approximation of microchannel. For calculations, rₛₒₗ, and rₐᵢᵣ, were set to be 50 μm and 1 mm respectively, thermal conductivities were k_{glass}=1.09 W m⁻¹° C.⁻¹ and kₐᵢᵣ=0.0256 W m⁻¹° C.⁻¹, and r_{glass} was kept variable.

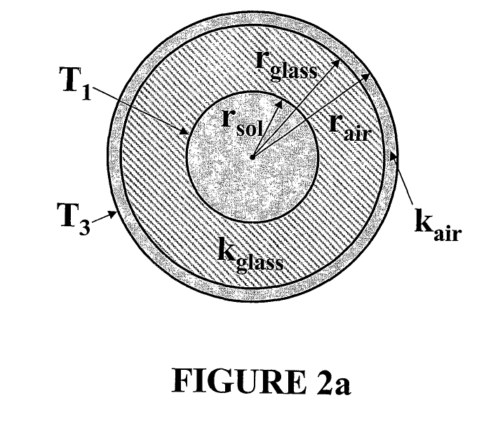

FIG. 2b is a graph showing the heat flow ratio (HFR) as a function of glass thickness. This approximation shows that etching the surrounding glass from 1.1 mm thickness to 0.05 mm thickness gives a 4.2-fold decrease in heat dissipation rate.

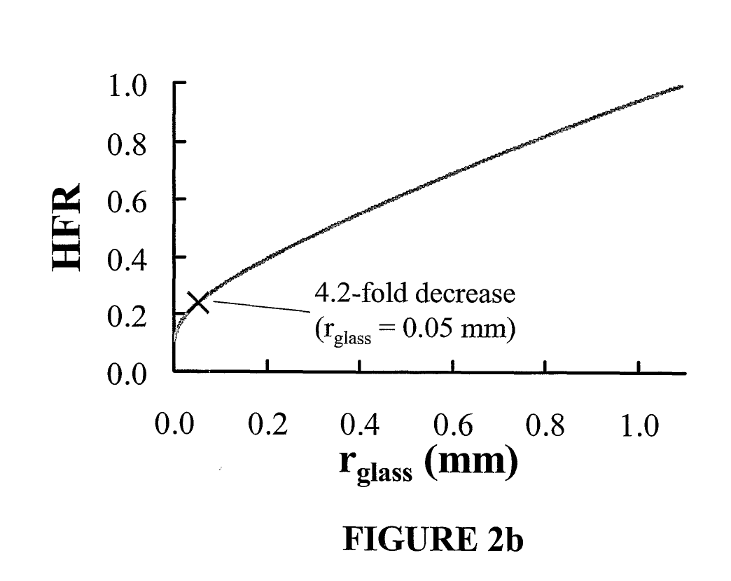

FIG. 3 is a schematic outlining a thermal mass removal method for glass microchips, utilizing the standard photolithography and wet etching protocol to pattern the bulk of the device along with the channel features.

FIG. 4 shows the photomask and etchant mask used in the process outlined in FIG. 3. This particular microfluidic or nanofluidic device consists of two reaction chambers that have been thermally isolated from the remainder of the chip by removal of surrounding glass.

FIG. 5a is a graph showing IR mediated heating and forced air cooling of two microfluidic devices made using the procedure shown in FIG. 3.

FIG. 5b is a graph showing the heating and cooling rates histogram derived from FIG. 5a.

FIG. 6 is a diagram showing an integrated microfluidic device having several thermally isolate regions.

FIG. 7 shows DNA amplification in the microfludic device made using the procedure shown in FIG. 3.

### DETAILED DESCRIPTION OF THE PREFERRED EMBODIMENTS

Microfluidic or nanofluidic devices typically include micromachined fluid networks. Fluid samples and reagents are brought into the device through entry ports and transported through channels to a reaction chamber, such as a thermally controlled reactor where mixing and reactions (e.g., synthesis, labeling, energy-producing reactions, assays, separations, or biochemical reactions) occur. The biochemical products may then be moved, for example, to an analysis module, where data is collected by a detector and transmitted to a recording instrument. The fluidic and electronic components are preferably designed to be fully compatible in function and construction with the reactions and reagents.

There are many formats, materials, and size scales for constructing microfluidic or nanofluidic devices. Common microfluidic or nanofluidic devices are disclosed in U.S. Pat. No. 6,692,700 to Handique et al.; U.S. Pat. No. 6,919,046 to O'Connor et al.; U.S. Pat. No. 6,551,841 to Wilding et al.; U.S. Pat. No. 6,630,353 to Parce et al.; U.S. Pat. No. 6,620,625 to Wolk et al.; and U.S. Pat. No. 6,517,234 to Kopf-Sill et al.; the disclosures of which are incorporated herein by reference. Typically, a microfludic device is made up of two or more substrates that are bonded together. Microscale components for processing fluids are disposed on a surface of one or more of the substrates. These microscale components include, but are not limited to, micro-reaction chambers, electrophoresis modules, microchannels, fluid reservoirs, detectors, valves, or mixers. When the substrates are bonded together, the microscale components are enclosed and sandwiched between the substrates. In many embodiments, at least inlet and outlet ports are engineered into the device for introduction and removal of fluid from the system. The microscale components can be linked together to form a fluid network for chemical and biological analysis. Those skilled in the art will recognize that substrates composed of silicon, glass, ceramics, polymers, metals and/or quartz are all acceptable in the context of the present invention. Further, the design and construction of the microfluidic or nanofluidic network vary depending on the analysis being performed and are within the ability of those skilled in the art.

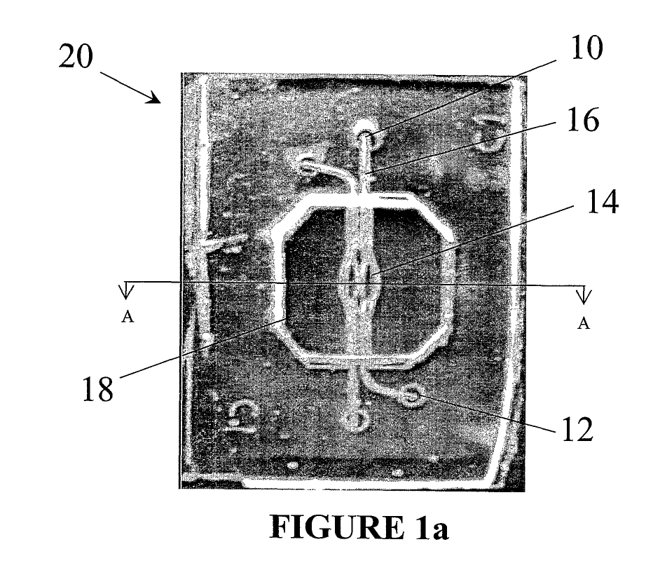

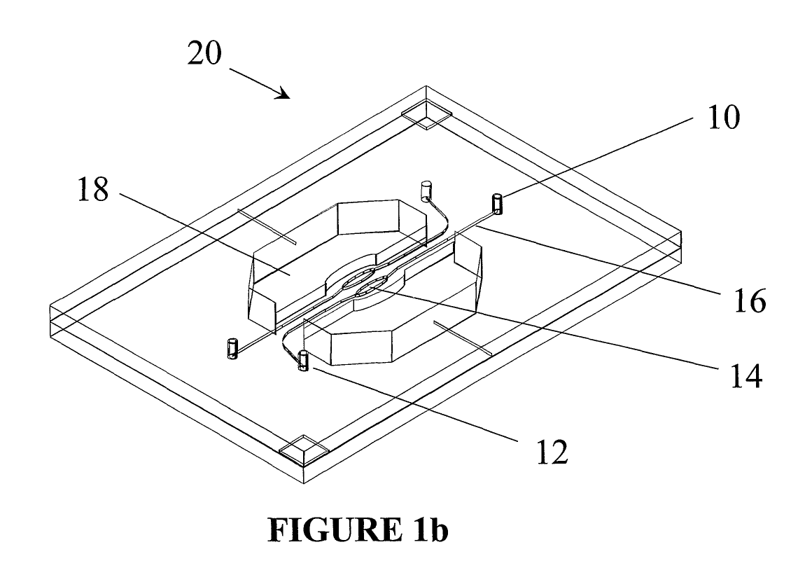

FIG. 1 shows a simple microfluidic or nanofluidic device 20 containing a fluid inlet 10 and fluid outlet 12 that are connected to a reaction chamber 14 by microchannels 16. The inlet 10 is used to introduce chemicals, solutions, and/or various reactants into the reaction chamber 14. Here, because the reaction chamber 14 requires rapid heating and cooling, the material immediately surrounding the reaction chamber 14 has been removed resulting in an empty space 18 and a bridge across the empty space 18 formed by the reaction chamber 14 and part of the channels 16. Because the removal of the material around the reaction chamber results in less thermal mass, increases in heating and cooling rates can be achieve for fluids inside the reaction chamber. It must be noted that the removal of the thermal mass must not, in any way, damage, interfere with, or render the microfluidic or nanofluidic network non-functional. The integrity of the microfluidic or nanofluidic network must be kept intact and functional, after the removal of the thermal mass.

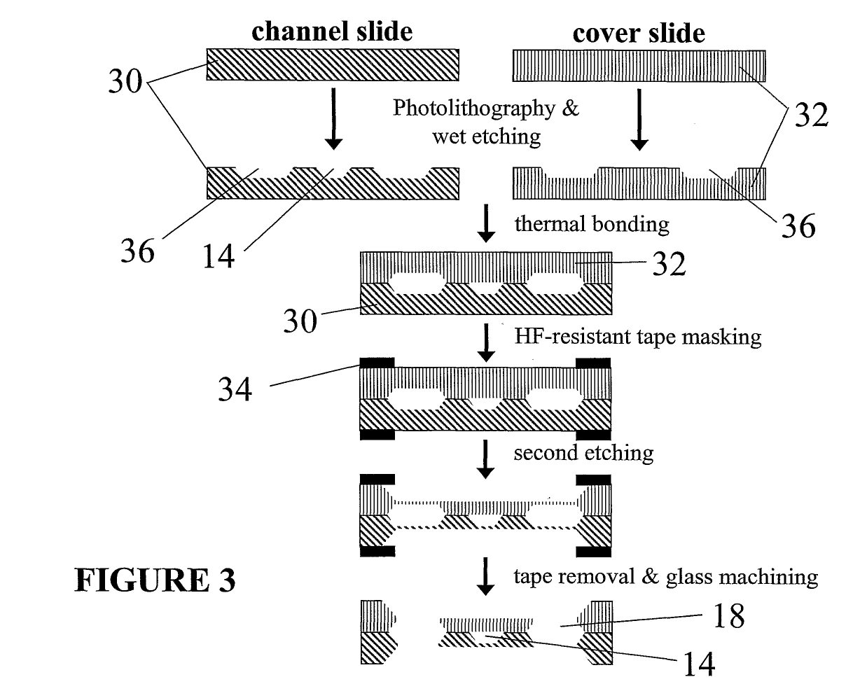

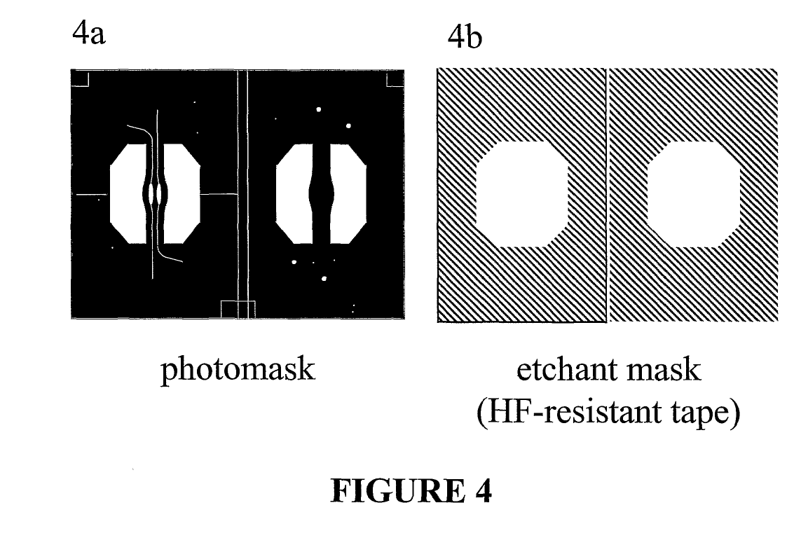

In a preferred embodiment, the microfluidic or nanofluidic device is made of glass through etching processes well-known in the art. This is shown in FIGS. 3 and 4. FIG. 3 shows cross section A-A of the device of FIG. 1; and FIG. 4 shows the masking required to accomplish the etching. The device of FIG. 1 is formed from two borofloat glass substrates, referred to herein as channel slide 30 and cover slide 32. The substrates 30 and 32 are masked (see FIG. 4a) and exposed to a UV source through the mask negative. The masks include thermal mass removal regions 36 on both channel slide 30 and cover slide 32 for this technique. The etched channel slide 30 and cover slide 32 are then bonded together (e.g., by thermal bonding) to form a microfluidic or nanofluidic device. Thermal mass is then preferably removed by further masking the top and bottom of the device (e.g., making tape, photolithography, etc.) (FIG. 4b), preferably HF resistant masking tape, and etched, preferably with HF, to the desired depth, which should be sufficiently shallow to maintain the structural integrity of the microscale components, such as the reaction chamber 14. After the second etching, a thin layer of glass encloses the mass removal regions 36, which can easily be machined to form the empty space 18. This results in the device of FIG. 1 having a reaction chamber 14 having less thermal mass, making it more amenable to rapid heating and cooling. In another embodiment, the mass removal regions 36 are made sufficiently deep, so that the second etching step is sufficient to expose the empty space 18 without having to machine the glass.

Although the removal of thermal mass is described in the second paragraph as being subsequent to the fabrication of the microchip, concurrent fabrication and thermal mass removal is also possible. For example, referring to FIG. 3, if the mass removal regions 36 of the slides are made deeper, the second etching step may not be necessary to further remove the thermal mass. Other mask designs to effect thermal mass removal concurrently with microchip fabrication are apparent to one skilled in the art.

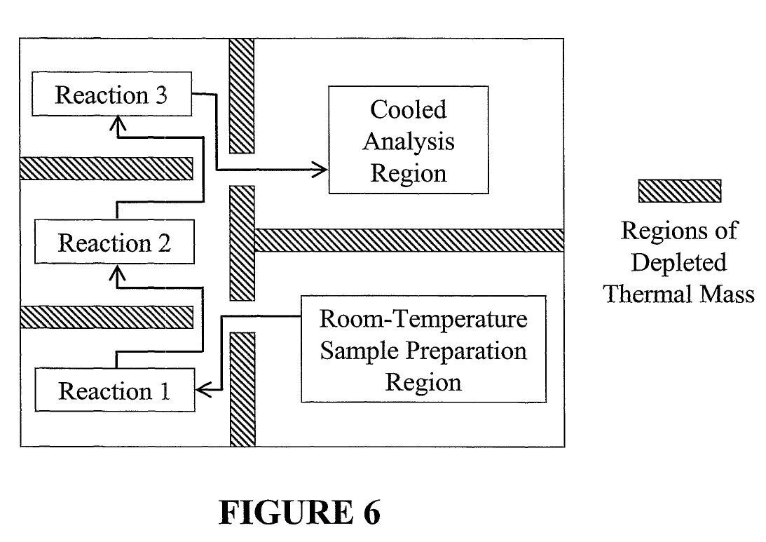

In another embodiment, the thermal mass can be removed to thermally isolate different regions on a microfluidic or nanofluidic device. FIG. 6 shows an example of such an embodiment wherein the microfluidic or nanofluidic device is divided into five different thermally isolated regions. In the device of FIG. 6, the sample preparation region operates at room temperature; the analysis region operates in a cooled environment; and reactions 1, 2, and 3, each operate at a specific temperature that can be the same or different from each other. Because the reactions 1, 2, and 3 are thermally separated, they are optimized for operation at different temperatures. However, if the reaction temperatures of reactions 1, 2, and 3 are all the same, then thermal mass removal to separate the three reactions is not required.

Although the preferred method disclosed herein uses glass, photolithography, and etching to prepare the microfludic device, other materials and methods to form the device and to remove thermal mass are also appropriate for the present invention (laser ablation, polymer molding, hot embossing, micromachining, physical/mechanical removal, etc.). Moreover, although FIGS. 1, 3, and 4 illustrate the reaction chamber as the selected area for rapid heating and/or cooling, other microscale components can also be selected. For example, a more complicated microfluidic or nanofluidic network may include multiple channels, reaction chambers, and fluid reservoir, not all of which are selected for rapid heating and/or cooling. In certain embodiments, certain channels and/or fluid reservoirs may be selected for rapid heating and/or cooling effected by removal of thermal mass surrounding the selected channels and/or fluid reservoirs. Further, in certain embodiments, it may be desirable to increase the thermal insulation in selected areas. This can be accomplished by depositing materials surrounding the selected areas to increase thermal insulation. The design and construction of the microfluidic or nanofluidic network vary depending on the specific analysis being performed and are within the ability of those skilled in the art.

In another embodiment, the removed thermal mass can be replaced with another material to achieve the desired thermal properties at selected regions. The refill material is selected based on the purpose and desired thermal property of the particular selected region. For example, a polymer can be removed and replaced with a metal to increase thermal conductivity in a selected region. On the other hand, if the thermal mass is removed to thermally isolate different regions on a microchip, then the region can be replaced with an insulative material.

In another embodiment, thermal properties of the substrate can be engineered to create a substrate with heterogeneous thermal properties throughout its volume. This is most preferable when used with a polymeric substrate. For example, the desired heat conductivity may be selectively changed in a selected region of the substrate by tuning the degree of cross-linking of a polymer in the selected region. Here, the desired thermal conductivity can be effected by varying the degree of cross-linking of the polymer.

The present invention is preferably used in conjunction with an apparatus for heating and cooling, such as that disclosed by U.S. Pat. No. 6,413,766 to Landers et al., the disclosure of which is incorporated herein by reference. Heating can be accomplished through any methods available, including, but is not limited to, optical energy, resistive heating, electrical elements, chemical heating, microwave heating, and contact heating. Preferably, optical energy is derived from an IR light source which emits light in the wavelengths known to heat water, which is typically in the wavelength range from about 0.775 μm to 7000 μm. For example, the infrared activity absorption bands of sea water are 1.6, 2.1, 3.0, 4.7 and 6.9 μm with an absolute maximum for the absorption coefficient for water at around 3 μm. The IR wavelengths are directed to the selected areas, and because the microfluidic or nanofluidic device is usually made of a clear or translucent material, the IR waves act directly upon the sample in the selected areas to cause heating. Although some heating of the sample might be the result of the reaction vessel itself absorbing the irradiation of the IR light, heating of the fluid in the selected area is primarily caused by the direct action of the IR wavelengths on the sample itself, because the thermal mass in the selected areas have sufficiently been removed.

Typically, the heating source will be an IR source, such as an IR lamp, an IR diode laser or an IR laser. An IR lamp is preferred, as it is inexpensive and easy to use. Preferred IR lamps are halogen lamps and tungsten filament lamps. Halogen and tungsten filament lamps are powerful, and can feed several reactions running in parallel. A tungsten lamp has the advantages of being simple to use and inexpensive, and can almost instantaneously (90% lumen efficiency in 100 msec) reach very high temperatures. A particularly preferred lamp is the CXR, 8V, 50 W tungsten lamp available from General Electric. That lamp is inexpensive and convenient to use, because it typically has all the optics necessary to focus the IR radiation onto the sample; no expensive lens system/optics will typically be required.

Heating can be effected in either one step, or numerous steps, depending on the desired application. For example, a particular methodology might require that the sample be heated to a first temperature, maintained at that temperature for a given dwell time, then heated to a higher temperature, and so on. As many heating steps as necessary can be included.

Similarly, cooling to a desired temperature can be effected in one step, or in stepwise reductions with a suitable dwell time at each temperature step. Cooling can be accomplished by any methods available including, but are not limited to, forced air, contact cooling, Peltier cooling, passive cooling, and chemical cooling. Positive cooling is preferably effected by use of a non-contact air source that forces air at or across the vessel. Preferably, that air source is a compressed air source, although other sources could also be used. It will be understood by those skilled in the art that positive cooling results in a more rapid cooling than simply allowing the vessel to cool to the desired temperature by heat dissipation. Cooling can be accelerated by contacting the selected areas with a heat sink comprising a larger surface than the selected areas themselves; the heat sink is cooled through the non-contact cooling source. The cooling effect can also be more rapid if the air from the non-contact cooling source is at a lower temperature than ambient temperature.

Accordingly, the non-contact cooling source should also be positioned remotely to the sample or reaction vessel, while being close enough to effect the desired level of heat dissipation. Both the heating and cooling sources should be positioned so as to cover the largest possible surface area on the sample vessel. The heating and cooling sources can be alternatively activated to control the temperature of the sample. It will be understood that more than one cooling source can be used.

Positive cooling of the reaction vessel dissipates heat more rapidly than the use of ambient air. The cooling means can be used alone or in conjunction with a heat sink. A particularly preferred cooling source is a compressed air source. Compressed air is directed at the selected areas when cooling of the sample is desired through use, for example, of a solenoid valve which regulates the flow of compressed air at or across the selected areas. The pressure of the air leaving the compressed air source can have a pressure of anywhere between 10 and 60 psi, for example. Higher or lower pressures could also be used. The temperature of the air can be adjusted to achieve the optimum performance in the thermocycling process. Although in most cases compressed air at ambient temperature can create enough of a cooling effect, the use of cooled, compressed air to more quickly cool the sample, or to cool the sample below ambient temperature might be desired in some applications.

A device for monitoring the temperature of the sample, and a device for controlling the heating and cooling of the sample, are also provided. Generally, such monitoring and controlling is accomplished by use of a microprocessor or computer programmed to monitor temperature and regulate or change temperature. An example of such a program is the Labview program (National Instruments, Austin, Tex.). Feedback from a temperature sensing device, such as a thermocouple or a remote temperature sensor, is sent to the computer. In one embodiment, the temperature sensing device provides an electrical input signal to the computer or other controller, which signal corresponds to the temperature of the sample. Preferably, the thermocouple, which can be coated or uncoated, is placed adjacent to the selected portions of the microfluidic or nanofluidic device where rapid heating and/or cooling is desired. Alternatively, the thermocouple can be placed directly into the microscale component, provided that the thermocouple does not interfere with the particular reaction or affect the thermocycling, and provided that the thermocouple used does not act as a significant heat sink. A suitable thermocouple for use with the present invention is constantan-copper thermocouple.

In a most preferred embodiment, temperature is monitored and controlled through a remote temperature sensing means. For example, a thermo-optical sensing device can be placed above an open reaction vessel containing the sample being thermocycled. Such a device can sense the temperature on a surface, here the surface of the sample, when positioned remotely from the selected areas.

The present methods and the resulting microfluidic or nanofluidic device are suitable for testing and incubation and treatment of biological and/or chemical samples typically analyzed in a laboratory or clinical diagnostic setting. The accuracy of the ability of the microfluidic or nanofluidic of the present invention to rapidly heat and/or cool makes it particularly suitable for use in nucleic acid replication by polymerase chain reaction (PCR). Any reaction that benefits from precise temperature control, rapid heating and cooling, continuous thermal ramping or other temperature parameters or variations can be accomplished using this method discussed herein. Other applications include, but are not limited to, the activation and acceleration of enzymatic reactions, the deactivation of enzymes, the treatment/incubation of protein-protein complexes, DNA-protein complexes, DNA-DNA complexes and complexes of any of these biomolecules with drugs and/or other organic or inorganic compounds to induce folding/unfolding and the association/dissociation of such complexes.

Without further description, it is believed that one of ordinary skill in the art can, using the preceding description and the following illustrative examples, make and utilize the present invention and practice the claimed methods. The following examples are given to illustrate the present invention. It should be understood that the invention is not to be limited to the specific conditions or details described in these examples.

### EXAMPLE 1

A microfluidic device was made according to the method outlined in FIG. 3. First, the borofloat glass with chrome and photoresist were exposed to the UV source through the mask negative (FIG. 4a) for 5 seconds. The mask included thermal mass removal regions on both the channel slide and cover slide. The exposed photoresist was then removed using a developer; and the remaining photoresist was hard-baked at 110° C. for 30 minutes. The exposed chrome was removed using chromium etchant. The glass was then etched using a solution of HF:HNO₃:H₂O (100:28:72) at a rate of approximately 2 μm/min. to the desired depth. The remaining photoresist was then removed using a stripper; and the remaining chrome was removed using chromium etchant. Using a diamond-tipped drill bit (1.1 mm diameter), reservoir holes were then drilled into the etched cover slide to align with the access channels on the channel slide. The channel and cover slides were cut to size and cleaned.

The glass plates were pressed together, placed between graphite coated ceramic plates, and placed in a high temperature furnace for bonding, where the furnace temperature was ramped to 550° C. at 8° C./min, then at 3° C./min to 670° C. The temperature was held at 670° C. for 3.5 hours before naturally cooling to room temperature to avoid cracking.

Etchant masks (FIG. 4b) were then created using HF-resistant tape and applied to the top and bottom of the bonded microchip. The chip was etched a second time using the 48% solution of HF at a rate of approximately 10 μm/min. Etching was stoppedjust before the glass was etched completely away from the thermal mass removal regions. The remaining thin sheets of glass were then broken away and cleaned from the thermal mass removal regions, leaving no glass in these regions. The process results in the microfluidic device of FIG. 1.

### EXAMPLE 2

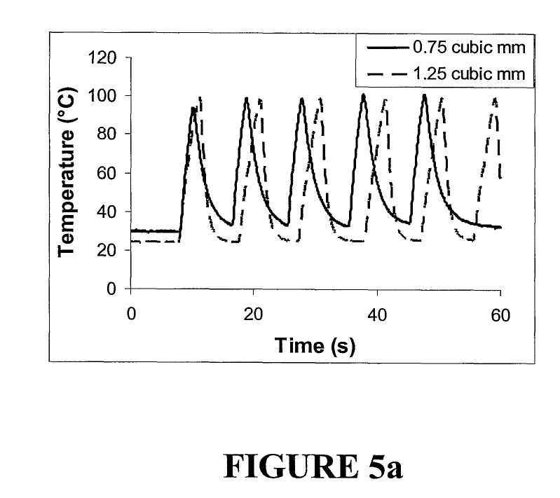

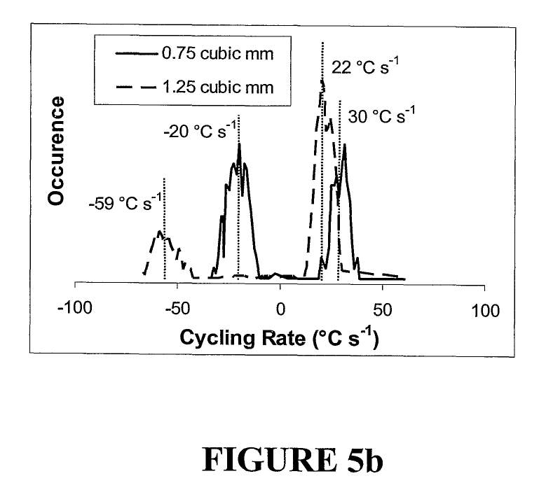

FIGS. 5a and b show the temperature profile and the heating and cooling rates of the for two different microfluidic devices made using the same method as Example 1. The solid line shows temperature profile and heating and cooling rates for a device having 0.75 mm³ of thermal mass remaining immediately around the reaction chamber; while the dashed line shows the same for a device having 1.25 mm³ remaining thermal mass. The device with more mass removed (less mass remaining) showed significant improvement in heating and cooling rates.

The following Table 1 compares heating rates of the microfluidic device of Example 1 with other chip configurations.

TABLE 1

| | Average heating | Average cooling |
|---|---|---|
| Configuration | rate (° C. s⁻¹) | rate (° C. s⁻¹) |
| A* | 1.0 | −1.0 |
| B | 30 | −20 |
| C | 22 | −59 |
| Capillary system | 65.0 | −20.0 |

A - Microfludic device with no thermal mass removal.
B - Microfludic device having 0.75 mm³ of thermal mass remaining immediately around the reaction chamber.
C - Microfludic device having 1.25 mm³ of thermal mass remaining immediately around the reaction chamber

Table 1 and FIG. 5 illustrated clearly the marked enhancement in IR heating and air cooling of solution that was possible in glass microchip reaction chambers using the present invention. The overall enhancement in rates was approximately 30-fold heating enhancement and 20-fold cooling enhancement. The cooling enhancement was not expected to be so drastic by simply removing thermal mass, but it was possible to move the air blower into close proximity with the microchamber and cool much faster than expected. The higher thermal mass removal (B) had higher heating rate, but lower cooling rate when compared to lower thermal mass removal (C). This suggests that it is possible to optimize the desired heating and cooling rates by controlling the amount of thermal mass removed.

These results have shown the great potential that this technique possesses for use in temperature-dependent microchip reactions, particularly PCR. However, the invention should be amenable to a wide variety of applications on any substrate. Not only was it shown that heating rates could be enhanced, but cooling rates as well (Table 1, FIG. 5) with the use of forced air. Enhanced heating/cooling could be applied to highly endothermic/exothermic reactions on microdevices for reaction rate enhancement. Cooling enhancement could even be applied to further reduction of Joule heating and allow even higher field strengths to be applied than was done by Jacobson et al.¹² as described previously. Heating rate enhancement could be applied to improve the detection limits of thermo-optical absorbance (TOA) detection on microchips. Any application on microdevices in which thermal control of solution conditions is required such as control of pH, viscosity, electroosmotic flow (EOF), etc., could feasibly benefit from this invention.

### EXAMPLE 3

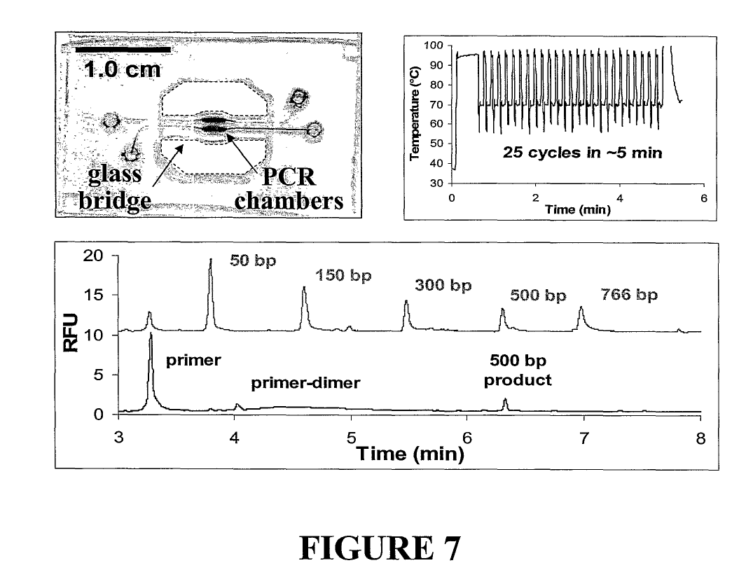

The microfluidic device made in Example 1 was used to perform DNA amplification through polymerase chain reaction (PCR) in the reaction chamber (see FIG. 7). The heating and cooling rates of the device were sufficiently fast to perform PCR in only 5 minutes. This is clearly a significant improvement over PCR using conventional methods, which take 1-3 hours to complete.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A method for making a microfluidic device comprising the steps of
providing at least one substrate of a first material;
fabricating a microfludic network in the substrate;
selecting regions on said substrate where a change in thermal property is desired; and
removing or adding material adjacent to the microfluidic network in said selected regions.

2. The method of claim 1, wherein material is removed if the desired change in thermal property is increased heating and/or cooling rate.

3. The method of claim 2, wherein the removing step is accomplished by etching, laser ablation, polymer molding, hot embossing, micromachining, or physical/mechanical removal.

4. The method of claim 2, wherein the removing step occurs after the fabricating step.

5. The method of claim 2, wherein the removing and fabricating steps occur concurrently.

6. The method of claim 2, further comprising the step of refilling the selected regions with a second material.

7. The method of claim 1, wherein the substrate is glass, polymer, ceramic, metal, silicon, or quartz.

8. The method of claim 1, wherein the microfluidic network contains at least a reaction chamber, microchannel, or fluid reservoir.

9. The method of claim 1, wherein material is added if the desired change in thermal property is increased heat insulation or heat transmission.

10. The method of claim 9, wherein the added material is different from the first material.

11. The method of claim 9, wherein the added material is the same as the first material.

12. The method of claim 9, wherein the added material is a metal.

13. A method for increasing cooling and/or heating rate of selected areas on a microfluidic device comprising the step of removing material adjacent to the selected areas.

14. The method of claim 13, wherein the removing step is accomplished by etching, laser ablation, polymer molding, hot embossing, or physical/mechanical removal.

15. The method of claim 13, wherein the microfludic device is made of glass, polymer, ceramic, metal, silicon, or quartz.

16. The method of claim 13, wherein the microfluidic network contains at least a reaction chamber, microchannel, or fluid reservoir.

17. A method for thermally isolating different regions of a microfluidic device comprising the steps of
identifying the regions to be isolated; and
removing material between the regions.

18. The method of claim 17, wherein the removing step is accomplished by etching, laser ablation, polymer molding, hot embossing, micromachining, or physical/mechanical removal.

19. The method of claim 17, wherein the microfludic device is made of glass, polymer ceramic, metal, silicon, or quartz.

20. The method of claim 17, wherein the microfluidic device contains at least a reaction chamber, microchannel, or fluid reservoir.

21. A method for performing analysis comprising the step of
providing a microfludic apparatus having at least a microfluidic network therein, at least a portion of the microfluidic network is thermally isolated by having material adjacent to said portion removed;
flowing at least a sample into said portion; and
heating and/or cooling said sample.

22. The method of claim 21, wherein the microfludic device is made of glass, polymer ceramic, metal, silicon, or quartz.

23. The method of claim 21, wherein the microfluidic network contains at least a reaction chamber, microchannel, or fluid reservoir.

24. The method of claim 21, wherein the sample contains DNA, RNA, proteins, or cells.

25. The method of claim 21, wherein the heating is accomplished by optical energy heating, resistive heating, electrical elements, chemical heating, microwave heating, and contact heating.

26. The method of claim 21, wherein cooling is accomplished by forced air cooling, contact cooling, Peltier cooling, passive cooling, or chemical cooling.

27. A microfluidic device comprising
at least one microscale component where material surrounding said component is removed or partially removed.

28. The microfluidic device of claim 27, wherein the microscale component is selected from the group consisting of reaction chambers, electrophoresis modules, microchannels, detectors, valves, and mixers.
